(19) United States
(12) Patent Application Publication
(10) Pub. No.: US 2016/0317019 A1
(43) Pub. Date: Nov. 3, 2016

(54) COMPUTER ASSISTED VISION DIAGNOSIS FOR LENS PRESCRIPTION

(71) Applicant: AiVision Pty Ltd, Rockdale (AU)

(72) Inventors: Desmond John Maddalena, Kirrawee (AU); Simon Grbevski, Kirrawee (AU)

(21) Appl. No.: 15/141,148

(22) Filed: Apr. 28, 2016

(30) Foreign Application Priority Data
Apr. 29, 2015 (AU) 2015 901 519

(51) Int. Cl. A61B 3/00; A61B 5/00; A61B 3/06; A61B 3/028; A61B 3/02

(52) U.S. Cl. CPC A61B 3/0025; A61B 3/0033; A61B 3/0058; A61B 3/028; A61B 3/022; A61B 3/066; A61B 5/0013; A61B 5/002; A61B 5/0022

(57) ABSTRACT

A computer-implemented method for vision diagnosis for lens prescription retrieves vision test data recorded from a computer assisted vision test of a patient. The vision test data of the patient is assessed for suitability for vision diagnosis. Where suitable, the vision test data of the patient is analyzed to display a representation of at least a part of the patient data. The analyzed part of the patient data is then matched with corresponding data from previously optometrically assessed and diagnosed vision data covering a wide range of optical conditions. A diagnosis of the vision of the patient is then established from the matching.

### PRIORITY CLAIM

The present application is a non-provisional of, claims priority to and the benefit of Australian Provisional Application Serial No. 2015 901 519, filed on Apr. 29, 2015, the entirety of which is incorporated herein by reference.

### REFERENCE TO RELATED PATENT APPLICATION

This application is related to Australian Patent Application No. 2014904932, filed Dec. 5, 2014 and entitled “Vision Testing for Astigmatism”, which is hereby incorporated by reference in its entirety as if fully set forth herein.

### TECHNICAL FIELD

The present invention relates generally to vision testing and, in particular, to a computer assisted vision diagnosis (CAVD) system configured to quickly diagnose visual problems utilizing remotely collected data from automated vision tests, combined with a CAVD module for rapid and improved diagnosis and corrective prescription generation with the legal certification from a vision specialist.

### BACKGROUND

International Patent Publication No. WO 02/00105 (PCT/AU01/00775), which issued for example as U.S. Pat. No. 7,367,675 and Australian Patent No. 2001267152, disclose a system for the testing of human eyesight. The system was substantially automated and could be performed by the human subject using an appropriately programmed general purpose computer and without the need for, or use of, one or more lenses interposed between the subject and a video display screen of the computer. The lensless system operated by executing one or more application programs on the computer and, through interaction between the subject and sequences of graphical images displayed on the display screen by the executing programs, the computer would record the subject's responses. The testing regimen firstly involved a setup phase which essentially calibrated the optical system formed by the subject and the display screen. Specific refractive vision tests performed included an acuity white E test, various astigmatism tests, near and distance acuity tests, a prefilter contrast test, a discrimination test, binocular tests, a saccades test as well as tests for cataracts, macular integrity, peripheral field and colour vision. The recording of the responses guided the execution of selected programs to capture detailed test data equivalent to that which would traditionally be recorded by an optometrist performing a traditional eyesight examination with the aid of interposed lenses.

The recorded test data would be processed by the local computer or remotely, for example at a server, to calculate at least one aspect of the visual functioning of the subject to thereby enable determination of an optical corrective lens prescription suitable for the subject. The processing and determination could be automated by computerized processing, assisted by a skilled optometrist, or performed in whole by the optometrist based on the test results. The approach nevertheless required the assistance of a professionally trained vision specialist to verify the results. The system enabled subjects at home or in community clinics, for example, to directly access and benefit from vision testing without a need to visit an optometrist or involving the use of expensive lenses. The verification step however was found to be time consuming and required specialist training.

Notwithstanding that professional oversight of test determination is considered essential in the medical and optical testing, the extent to which processing can be automated or streamlined can reveal significant progress in terms of reliability, time to diagnosis and thus savings in cost and time of product delivery, such as supply of prescription lenses to a remote subject.

### SUMMARY

It is desirable to provide for the substantially automated processing of vision test data for the determination of an optical lens prescription.

According to a first aspect of the present disclosure, there is provided a computer-implemented method for vision diagnosis for lens prescription, comprising:

retrieving vision test data recorded from a computer assisted vision test of a patient;

assessing the vision test data of the patient for suitability for vision diagnosis;

analyzing the vision test data of the patient to display a representation of at least a part of the patient data;

matching the analyzed part of the patient data with corresponding data from previously optometrically assessed and diagnosed vision data covering a wide range of optical conditions; and

establishing a diagnosis of the vision of the patient from the matching.

Desirably the assessing comprises validating patient data and validating patient pathology such that where either such validation fails, the method terminates. The validating of the patient data may comprise validating data associated with the capture of the vision test data of the patient, the capture data being at least one of test date, screen size, screen colours, and age range. The validating of the patient pathology may comprise assessing patient answers to qualitative questions regarding at least one of lazy eye, surgery, cataracts, glaucoma and macular degeneration.

Preferably the matching comprises displaying a representation of a corresponding part of the previously optometrically assessed and diagnosed patient data with the displayed representation of the analyzed part of the patient data.

Advantageously the analyzing and matching comprise displaying, in a graphical user interface, adjacent representations of analysed current patient data and previously optometrically assessed and diagnosed patient data for at least one indicator pattern associated with the vision test data.

In one example, the indicator pattern is a type profile having matching types selected from the group consisting of big hyperope, hyperope, mid, myope, big myope, astigmatic myope, astigmatic hyperope, pathological hyperope, and pathological myope. In another example, the indicator pattern is associated with a Growing C test. In another example, the indicator pattern is associated with a Prelim test. In another example, the indicator pattern is associated with a Contrast test. In another example, the indicator pattern is associated with a Near test. In another example, the indicator pattern is associated with a chromic test.

Preferably the establishing of the diagnosis comprises displaying in the graphical user interface values for at least each of spherical power, cylindrical power, and astigmatic angle, for each patient eye. Desirably, this may further comprise displaying in the graphical user interface a value for additional optical power for near reading component for each patient eye.

In specific implementations the vision test data comprises computer assisted vision test data of a plurality of patients, and the method may further comprise dividing the test data associated with the patients into a plurality of groups based upon measured values of visual acuity, and processing patient data associated with each group as a stream. Advantageously:

a first group comprises patients with visual acuity in the range −1.5 to +1.5 dioptres, and who have only minor astigmatism, generally less than 0.5 dioptres;

a second group comprises patients with visual acuity in the range −1.5 to −4 and +1.5 to 4 dioptres; and

a third group comprises patients with visual acuity problems outside the range −1.5 to −4 and +1.5 to 4 dioptres.

According to another aspect of the present disclosure there is provided a system for assisted vision diagnosis for lens prescription, comprising:

a database of previously optometrically assessed and diagnosed vision data covering a wide range of optical conditions establishing a plurality of visual indicator patterns;

a vision test data set associated with a patient recorded from a computer assisted vision test of the patient;

a processor associated with a program, the program being executable by the processor to:
retrieve the vision test data of the patient;
assess the vision test data set of the patient for suitability for vision diagnosis;
analyze the vision test data set of the patient to display a representation of at least a part of the patient test data;
match the analyzed part of the patient data with corresponding data from the database of previously optometrically assessed and diagnosed vision data; and
establish a diagnosis of the vision of the patient from the matching.

Other aspects are also disclosed.

### BRIEF DESCRIPTION OF THE FIGURES

At least one embodiment of the present invention will now be described with reference to the drawings and appendices, in which:

FIGS. 1A and 1B form a schematic block diagram of a general purpose computer system upon which arrangements described can be practiced;

FIG. 2 is a flowchart of a method of optical test results processing;

FIGS. 3 to 5 illustrate introductory screen shots of a graphical user interface (GUI) for optical test results processing;

FIG. 6 is a screen shot of an analysis interface screen of the GUI;

FIGS. 7 and 8 show respectively exemplary detail of the data output and results portions of the screen of FIG. 6;

FIG. 9 shows an exemplary detail of a type profile of a portion of the screen of FIG. 6;

FIGS. 10A to 10I show exemplary type matches that may be viewed in the portion of FIG. 9;

FIG. 11A shows exemplary Growing C test results that may be viewed in the portion of FIG. 9;

FIG. 11B shows exemplary Prelim test results that may be viewed in the portion of FIG. 9;

FIG. 11C shows exemplary Contrast test results that may be viewed in the portion of FIG. 9;

FIG. 11D shows exemplary Near test results that may be viewed in the portion of FIG. 9;

FIG. 11E shows exemplary Chromic test results that may be viewed in the portion of FIG. 9;

FIG. 11F shows an exemplary diagnosis result that may be viewed in the portion of FIG. 9;

FIG. 12 is a flowchart of a preferred patient data validation step;

FIG. 13 is a flowchart of a preferred pathology validation step;

FIG. 14 shows an exemplary Growing C plot; and

FIG. 15 shows an exemplary Prelim Power curve.

### DETAILED DESCRIPTION

In this description, the terms “client”, “subject” and “patient” are used interchangeably and mean the person undergoing or having undergone vision testing and for which optical lens prescription(s) are desired. Similarly “user”, “diagnostician”, and “practitioner” are used interchangeably and mean a person who implements or operates apparatus according to the present disclosure to diagnose the optical lens prescription(s) for the first mentioned person who underwent vision testing.

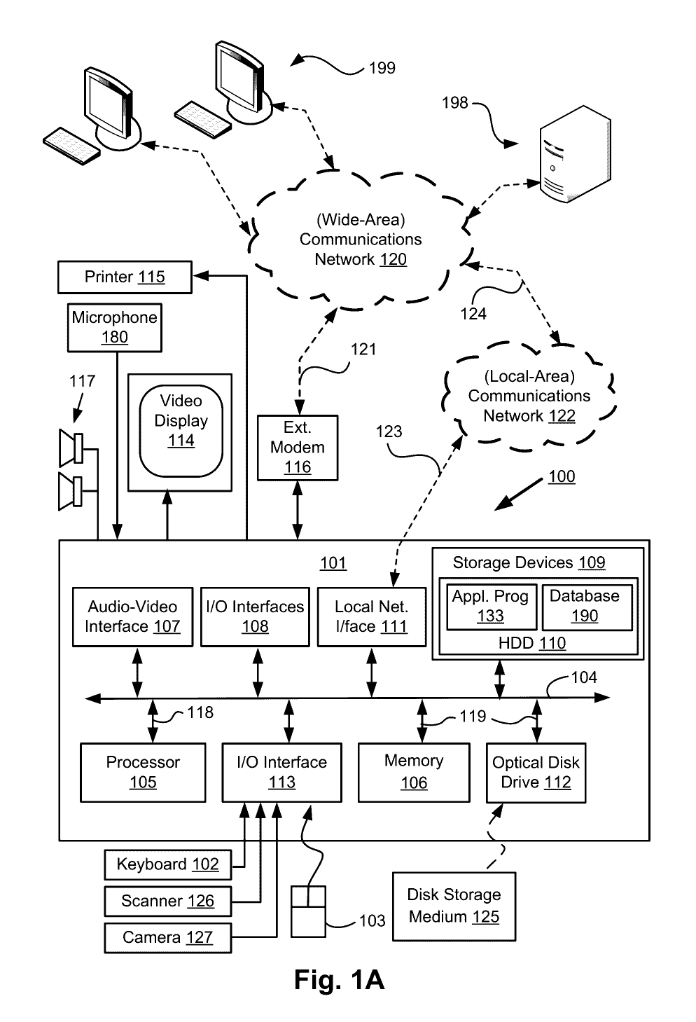

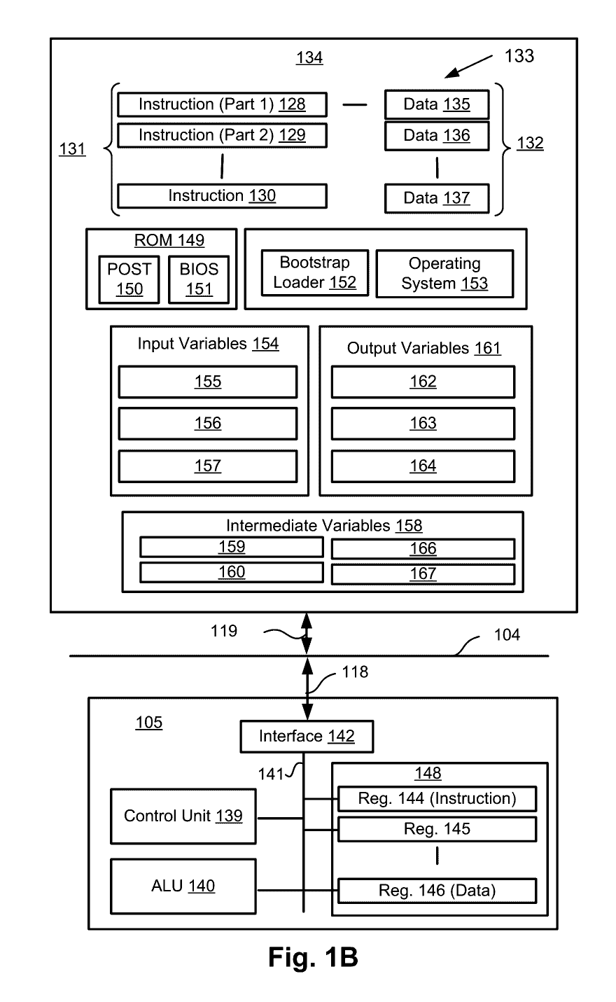

FIGS. 1A and 1B depict a general-purpose computer system 100, upon which the various arrangements described can be practiced.

As seen in FIG. 1A, the computer system 100 includes: a computer module 101; input devices such as a keyboard 102, a mouse pointer device 103, a scanner 126, a camera 127, and a microphone 180; and output devices including a printer 115, a display device 114 and loudspeakers 117. An external Modulator-Demodulator (Modem) transceiver device 116 may be used by the computer module 101 for communicating to and from a communications network 120 via a connection 121. The communications network 120 may be a wide-area network (WAN), such as the Internet, a cellular telecommunications network, or a private WAN. Where the connection 121 is a telephone line, the modem 116 may be a traditional “dial-up” modem. Alternatively, where the connection 121 is a high capacity (e.g., cable) connection, the modem 116 may be a broadband modem. A wireless modem may also be used for wireless connection to the communications network 120. The networks 120 and 122 permit interconnection to one or more remote (client) computer terminals 199 at which vision testing can be performed upon corresponding subjects/patients and which are configured to interact with a testing program. The testing program operates in accordance with the disclosure of the aforementioned International Patent Publication No. WO 02/00105 and Australian Patent Application No. 2014904932 and can be executed upon the computer module 101, or alternatively upon a server computer 198 remote from both the computer 101 and the client computers 199, via interaction with the respective client computer 199.

The computer module 101 typically includes at least one processor unit 105, and a memory unit 106. For example, the memory unit 106 may have semiconductor random access memory (RAM) and semiconductor read only memory (ROM). The computer module 101 also includes an number of input/output (I/O) interfaces including: an audio-video interface 107 that couples to the video display 114, loudspeakers 117 and microphone 180; an I/O interface 113 that couples to the keyboard 102, mouse 103, scanner 126, camera 127 and optionally a joystick or other human interface device (not illustrated); and an interface 108 for the external modem 116 and printer 115. In some implementations, the modem 116 may be incorporated within the computer module 101, for example within the interface 108. The computer module 101 also has a local network interface 111, which permits coupling of the computer system 100 via a connection 123 to a local-area communications network 122, known as a Local Area Network (LAN). As illustrated in FIG. 1A, the local communications network 122 may also couple to the wide network 120 via a connection 124, which would typically include a so-called “firewall” device or device of similar functionality. The local network interface 111 may comprise an Ethernet circuit card, a Bluetooth™ wireless arrangement or an IEEE 802.11 wireless arrangement; however, numerous other types of interfaces may be practiced for the interface 111.

The I/O interfaces 108 and 113 may afford either or both of serial and parallel connectivity, the former typically being implemented according to the Universal Serial Bus (USB) standards and having corresponding USB connectors (not illustrated). Storage devices 109 are provided and typically include a hard disk drive (HDD) 110. Other storage devices such as a floppy disk drive and a magnetic tape drive (not illustrated) may also be used. An optical disk drive 112 is typically provided to act as a non-volatile source of data. Portable memory devices, such optical disks (e.g., CD-ROM, DVD, Blu-ray Disc™), USB-RAM, portable, external hard drives, and floppy disks, for example, may be used as appropriate sources of data to the system 100.

The components 105 to 113 of the computer module 101 typically communicate via an interconnected bus 104 and in a manner that results in a conventional mode of operation of the computer system 100 known to those in the relevant art. For example, the processor 105 is coupled to the system bus 104 using a connection 118. Likewise, the memory 106 and optical disk drive 112 are coupled to the system bus 104 by connections 119. Examples of computers on which the described arrangements can be practised include IBM-PC's and compatibles, Sun Sparcstations, Apple Mac™ or a like computer systems.

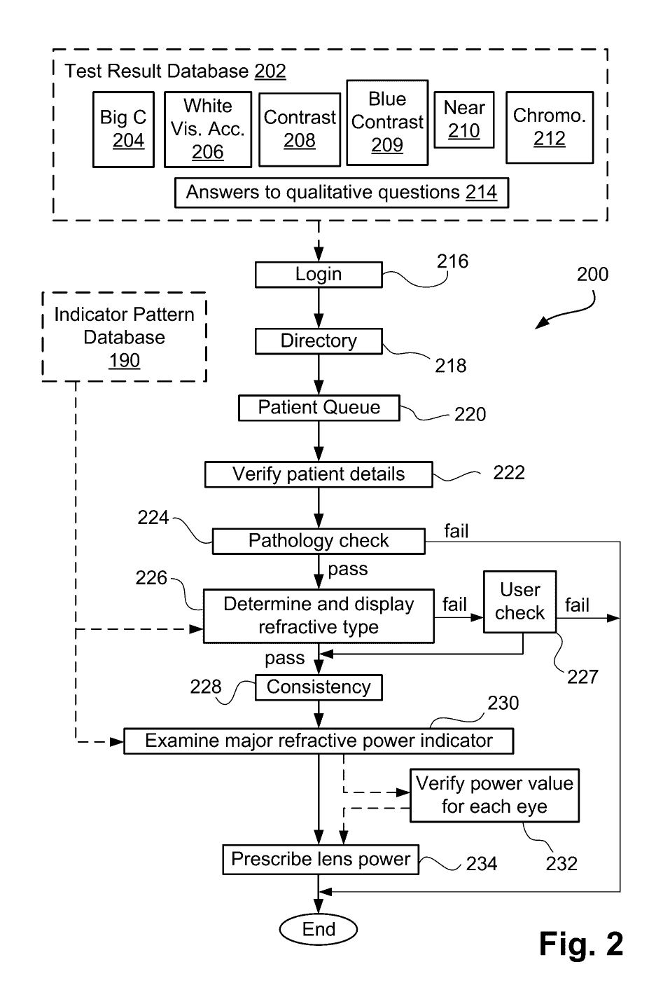

The methods of vision test data analysis may be implemented using the computer system 100 wherein the processes of FIGS. 2 to 11F, to be described, may be implemented as one or more software application programs 133 executable within the computer system 100. In particular, the steps of the methods of vision test data analysis are effected by instructions 131 (see FIG. 1B) in the software 133 that are carried out within the computer system 100. The software instructions 131 may be formed as one or more code modules, each for performing one or more particular tasks. The software may also be divided into two separate parts, in which a first part and the corresponding code modules performs the analysis methods and a second part and the corresponding code modules manage a user interface between the first part and the user.

The software may be stored in a computer readable medium, including the storage devices described below, for example. The software is loaded into the computer system 100 from the computer readable medium, and then executed by the computer system 100. A computer readable medium having such software or computer program recorded on the computer readable medium is a computer program product. The use of the computer program product in the computer system 100 preferably effects an advantageous apparatus for vision test data analysis.

The software 133 is typically stored in the HDD 110 or the memory 106. The software is loaded into the computer system 100 from a computer readable medium, and executed by the computer system 100. Thus, for example, the software 133 may be stored on an optically readable disk storage medium (e.g., CD-ROM) 125 that is read by the optical disk drive 112. A computer readable medium having such software or computer program recorded on it is a computer program product. The use of the computer program product in the computer system 100 preferably effects an apparatus for vision test data analysis.

In some instances, the application programs 133 may be supplied to the user encoded on one or more CD-ROMs 125 and read via the corresponding drive 112, or alternatively may be read by the user from the networks 120 or 122. Still further, the software can also be loaded into the computer system 100 from other computer readable media. Computer readable storage media refers to any non-transitory tangible storage medium that provides recorded instructions and/or data to the computer system 100 for execution and/or processing. Examples of such storage media include floppy disks, magnetic tape, CD-ROM, DVD, Blu-ray Disc™, a hard disk drive, a ROM or integrated circuit, USB memory, a magneto-optical disk, or a computer readable card such as a PCMCIA card and the like, whether or not such devices are internal or external of the computer module 101. Examples of transitory or non-tangible computer readable transmission media that may also participate in the provision of software, application programs, instructions and/or data to the computer module 101 include radio or infra-red transmission channels as well as a network connection to another computer or networked device, and the Internet or Intranets including e-mail transmissions and information recorded on Websites and the like.

The second part of the application programs 133 and the corresponding code modules mentioned above may be executed to implement one or more graphical user interfaces (GUIs) to be rendered or otherwise represented upon the display 114. Through manipulation of typically the keyboard 102 and the mouse 103, a user of the computer system 100 and the application may manipulate the interface in a functionally adaptable manner to provide controlling commands and/or input to the applications associated with the GUI(s). Other forms of functionally adaptable user interfaces may also be implemented, such as an audio interface utilizing speech prompts output via the loudspeakers 117 and user voice commands input via the microphone 180.

FIG. 1B is a detailed schematic block diagram of the processor 105 and a “memory” 134. The memory 134 represents a logical aggregation of all the memory modules (including the HDD 109 and semiconductor memory 106) that can be accessed by the computer module 101 in FIG. 1A.

When the computer module 101 is initially powered up, a power-on self-test (POST) program 150 executes. The POST program 150 is typically stored in a ROM 149 of the semiconductor memory 106 of FIG. 1A. A hardware device such as the ROM 149 storing software is sometimes referred to as firmware. The POST program 150 examines hardware within the computer module 101 to ensure proper functioning and typically checks the processor 105, the memory 134 (109, 106), and a basic input-output systems software (BIOS) module 151, also typically stored in the ROM 149, for correct operation. Once the POST program 150 has run successfully, the BIOS 151 activates the hard disk drive 110 of FIG. 1A. Activation of the hard disk drive 110 causes a bootstrap loader program 152 that is resident on the hard disk drive 110 to execute via the processor 105. This loads an operating system 153 into the RAM memory 106, upon which the operating system 153 commences operation. The operating system 153 is a system level application, executable by the processor 105, to fulfil various high level functions, including processor management, memory management, device management, storage management, software application interface, and generic user interface.

The operating system 153 manages the memory 134 (109, 106) to ensure that each process or application running on the computer module 101 has sufficient memory in which to execute without colliding with memory allocated to another process. Furthermore, the different types of memory available in the system 100 of FIG. 1A must be used properly so that each process can run effectively. Accordingly, the aggregated memory 134 is not intended to illustrate how particular segments of memory are allocated (unless otherwise stated), but rather to provide a general view of the memory accessible by the computer system 100 and how such is used.

As shown in FIG. 1B, the processor 105 includes a number of functional modules including a control unit 139, an arithmetic logic unit (ALU) 140, and a local or internal memory 148, sometimes called a cache memory. The cache memory 148 typically includes a number of storage registers 144-146 in a register section. One or more internal busses 141 functionally interconnect these functional modules. The processor 105 typically also has one or more interfaces 142 for communicating with external devices via the system bus 104, using a connection 118. The memory 134 is coupled to the bus 104 using a connection 119.

The application program 133 includes a sequence of instructions 131 that may include conditional branch and loop instructions. The program 133 may also include data 132 which is used in execution of the program 133. The instructions 131 and the data 132 are stored in memory locations 128, 129, 130 and 135, 136, 137, respectively. Depending upon the relative size of the instructions 131 and the memory locations 128-130, a particular instruction may be stored in a single memory location as depicted by the instruction shown in the memory location 130. Alternately, an instruction may be segmented into a number of parts each of which is stored in a separate memory location, as depicted by the instruction segments shown in the memory locations 128 and 129.

In general, the processor 105 is given a set of instructions which are executed therein. The processor 105 waits for a subsequent input, to which the processor 105 reacts to by executing another set of instructions. Each input may be provided from one or more of a number of sources, including data generated by one or more of the input devices 102, 103, data received from an external source across one of the networks 120, 122, data retrieved from one of the storage devices 106, 109 or data retrieved from a storage medium 125 inserted into the corresponding reader 112, all depicted in FIG. 1A. The execution of a set of the instructions may in some cases result in output of data. Execution may also involve storing data or variables to the memory 134.

The disclosed vision test data analysis arrangements use input variables 154, which are stored in the memory 134 in corresponding memory locations 155, 156, 157. The arrangements produce output variables 161, which are stored in the memory 134 in corresponding memory locations 162, 163, 164. Intermediate variables 158 may be stored in memory locations 159, 160, 166 and 167.

Referring to the processor 105 of FIG. 1B, the registers 144, 145, 146, the arithmetic logic unit (ALU) 140, and the control unit 139 work together to perform sequences of micro-operations needed to perform “fetch, decode, and execute” cycles for every instruction in the instruction set making up the program 133. Each fetch, decode, and execute cycle comprises:

(i) a fetch operation, which fetches or reads an instruction 131 from a memory location 128, 129, 130;

(ii) a decode operation in which the control unit 139 determines which instruction has been fetched; and

(iii) an execute operation in which the control unit 139 and/or the ALU 140 execute the instruction.

Thereafter, a further fetch, decode, and execute cycle for the next instruction may be executed. Similarly, a store cycle may be performed by which the control unit 139 stores or writes a value to a memory location 132.

Each step or sub-process in the processes of FIGS. 2 to 11E is associated with one or more segments of the program 133 and is performed by the register section 144, 145, 146, the ALU 140, and the control unit 139 in the processor 105 working together to perform the fetch, decode, and execute cycles for every instruction in the instruction set for the noted segments of the program 133.

### Overview

The present disclosure provides a computerised and substantially automated diagnostic system which uses remote eye test data obtained from systems operating according to the aforementioned patent documents, together with a database of new data from subsequent clinical studies, to improve the accuracy and speed of the lens prescription diagnosis and subsequent corrective lens calculations. The present inventors have found that both the prescription accuracy and speed to diagnosis is further improved by preferably staging the remotely obtained vision test data into a number of streams or groups. There should be at least two such groups and preferably three groups. The speed of diagnosis is important as it reduces professional labour costs and improves the commercial viability of the vision testing arrangements.

FIG. 2 shows a flowchart of a CAVD method 20X), which is desirably implemented in software as the application program 133, to analyse vision test data received from one or more and preferably many of the client computers 199 and to aid a practitioner in the specification of a lens prescription for a subject associated with a corresponding set of test results. In a preferred implementation, the method 200 operates on a set of eye test data received from a remote eye test computer program that operates for example as described in the aforementioned patent documents, where the set of eye test data is stored in a temporary database 202, formed for example within the memory 106.

The method 200 retrieves the raw data from the temporary result database 202 and decrypts and decodes that raw test data into test data parameters that can be used in various optical calculations.

The parameter set stored in the database 202 is desirably a linear chain of data that relates to the individual tests carried out during the vision test stage. The parameter set includes data about the client's name, date of test, test image size at calibration, colour intensity at calibration, Big C test data, astigmatic test data, contrast pattern test data, growing C test data, the colour contrast test data, near test data. These various tests are as described in the aforementioned International Patent Publication No. WO 02/00105.

Some of parameters are used to validate the test data, while others are used to detect pathology,

Some of the parameters are used to indicate the refractive status of the patient, thereby allowing patients to be staged into groups or streams by which their corresponding test data can be processed at different speeds with increased efficiency which operates to enhance speed of the diagnosis. Other optical parameters allow the calculation of spherical optical power in four different ways, and astigmatic power and axis in two different ways, to ensure reliability and accuracy.

The processing and diagnosis is preferably structured as three steps. A first step uses test data 610 (to be described) and is desirably composed of two parts—the first being the validation step which validates the accuracy of the test data set; and the second part which looks for pathology test data 710 (to be described) and current health which may interfere with an accurate and correct diagnosis. A failure in this first step will abort the diagnosis.

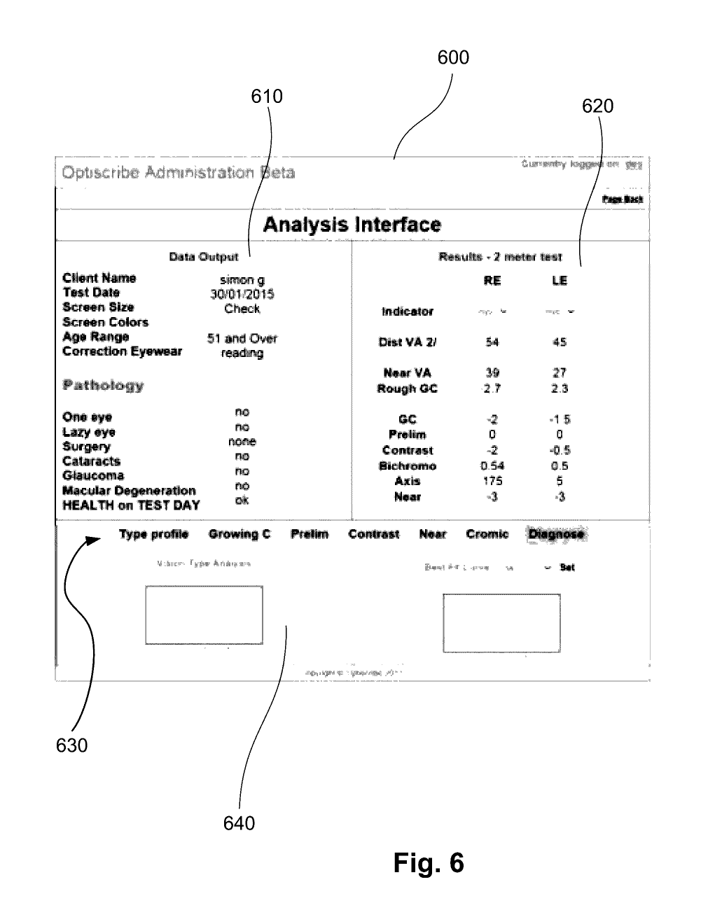

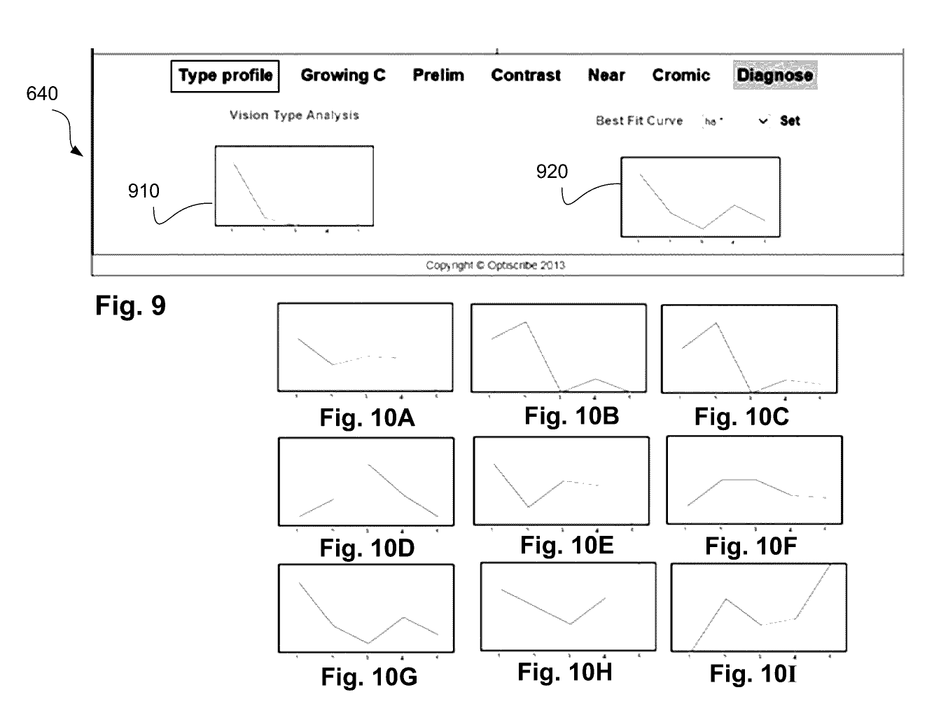

The second step is to stage the diagnosis into three streams or groups to enhance and assist the speed and reliability of the diagnosis and to reduce operator fatigue. This is used where batches of patient test result data are available. This involves the use of indicator patterns, shown for example in FIG. 6 as test data 630 and 640, and FIG. 9, test data 910 and 920 and FIGS. 10A to 10I (to be described).

When carrying out an analysis of data sets, a graph or other visual representation which gives an overall mental image of the client's visual data set is used as a valuable way to start an analysis. The indicator patterns were developed to assist this operation.

The indicator patterns are derived from a database, stored for example within the HDD 110 and illustrated in FIG. 1A as the database 190, and formed from previously optometrically assessed and diagnosed vision data covering a wide range of optical conditions. The database 190 may store simply the prior diagnosed patient data, which can be interpreted by the application 133, like that of the current patient data, to provide a visual indicator pattern of that prior data within the GUI. Alternatively the database 190 may store just the prior diagnosed patient indicator patterns. In a preferred implementation, the database 190 formed within the HDD 110 stores both the prior diagnosed patient data and the corresponding indicator patterns. In a specific implementation, the database 190 is formed as two databases, one for raw data associated with optometrically assessed vision tests covering a wide range of optical conditions, and another to house diagnosed final results with corresponding indicator patterns. This separation makes it harder for people to gain illicit entry to the final patient data, allowing improved patient data security.

The indicator patterns provide, via graphs, charts, histograms and the like, a visual representation of known diagnosed optical conditions by which a visual comparison with corresponding visual representations of the current patient data may be compared, and hopefully matched. A trained analyst can, by examination and comparison of the indicator patterns, ascertain the type of problem presented by the current patient, the severity and pathology of the patient's optical condition, and thus speed up the diagnosis.

As will be described, the indicator pattern is composed of four indices, one each for hyperopia, normality, myopia and severity shown graphed from left to right in the graphical representation.

The indices are calculated based on a statistical study of the diagnostic importance of the patient's answers to a graded set of visual history questions which are presented to the patients in common language readily understood by the patient that takes into account their ethnicity and likely level of knowledge. The answers 214 of the database 202 represent the inputs to the index determination.

The visual history questions are rated as to whether the answers would normally indicate hyperopia, normality or myopia. The responses and answers are summed to give an indication of severity. Using this approach it is possible to create a set of four-index patterns with different shapes that can be used to directly identify the patient's visual problem from the patient's point of view.

The indicator patterns allow automated staging of the analysis into the preferred three streams, Group A, Group B, and Group C.

Group A preferably represents those patients/clients who have visual problems within the range −1.5 to +1.5 dioptres, who have only minor astigmatism, generally less than 0.5 dioptres, and who are generally younger people under the age of about 40 with no pathology problems. Manual diagnosis of these clients traditionally took 3-5 minutes. Computer assisted diagnosis according to the present disclosure takes less than 1 minute per patient.

Group B preferably represents those patients/clients who have visual acuity problems in the range −1.5 to −4 and +1.5 to 4 dioptres. Such patients often have greater astigmatic powers from 0.75-3 dioptres and often have associated or suspected minor underlying visual pathology. Manual diagnosis this group typically takes longer, quite often in the vicinity of 6-10 minutes. Computer assisted diagnosis according to the present disclosure generally takes less than 5 minutes per patient.

Group C represents those patients/clients are those generally outside the +/−4 dioptre range, (or +/−6 dioptres when tested at a distance of 1 metre) have suspected or known pathology, and are considered too difficult to diagnose from remote vision testing data. These clients are usually automatically rejected and advised to see a vision specialist as their cases were too complicated for remote diagnosis.

In an alternative implementation, Groups A and B may be combined into a single group for automated processing, and the second group (Group C) excluded from automated processing.

If the data is found to be inconsistent, indicating the test conditions were not acceptable or the test conditions were varied during the test, then a “Fail” condition is triggered.

If the “Fail” condition is indicated then it would be up to the discretion of the reviewing vision professional as to whether to accept the data as being reliable and continue analysis by disregarding the “Fail” condition, or to ask patient to repeat the test.

The third step involves the initial analysis. This step is carried out using the data displayed in FIG. 8 test data 620. In this step the user of the method 200 (the diagnostician) utilises the data which is displayed in conventional visual acuity terminology such as 6/6 for a normal sight decreasing to 6/60 indicating refractive blindness. This allows the diagnosis to relate the values to a conventional acuity diagnosis. The diagnostician can then determine whether the computerised analysis and the displayed refractive value is consistent with the expected acuity to refractive power norms.

If the display indicates that the analysis was from a Group A patient then the diagnostic reliability of the results is high and the diagnostician can confidently rely upon the computer assisted diagnosis to be highly likely to be correct. The diagnostician can proceed to confirm a lens prescription to be issued.

If the display indicates that the analysis was from a Group B patient with lower reliability then the diagnostician may then manually examine the data using the graphic interface 620, to examine the patient's data in comparison to data from other matching client clinical data sets. This ability to instantly match the patient's data sets to other known clinical sets increases confidence and reliability to make a more accurate diagnosis of the patient's refractive status. If necessary the diagnostician can manually make adjustments as appropriate to the lens prescription.

The matching of the data sets is done by using a least squares match algorithm comparing the optical power results from astigmatism, growing C, contrast pattern, colour contrast pattern and near power results, thereby giving similar data sets obtained in a clinical environment by an optometrist.

### Vision Test Method

As seen in FIG. 2, data from the remote vision tests is stored in the database 202, for example formed within the memory 106, which is typically remove from the client computers 199 at which actual test data is obtained. The presently described analytical machine process 200 utilises the following data streams:

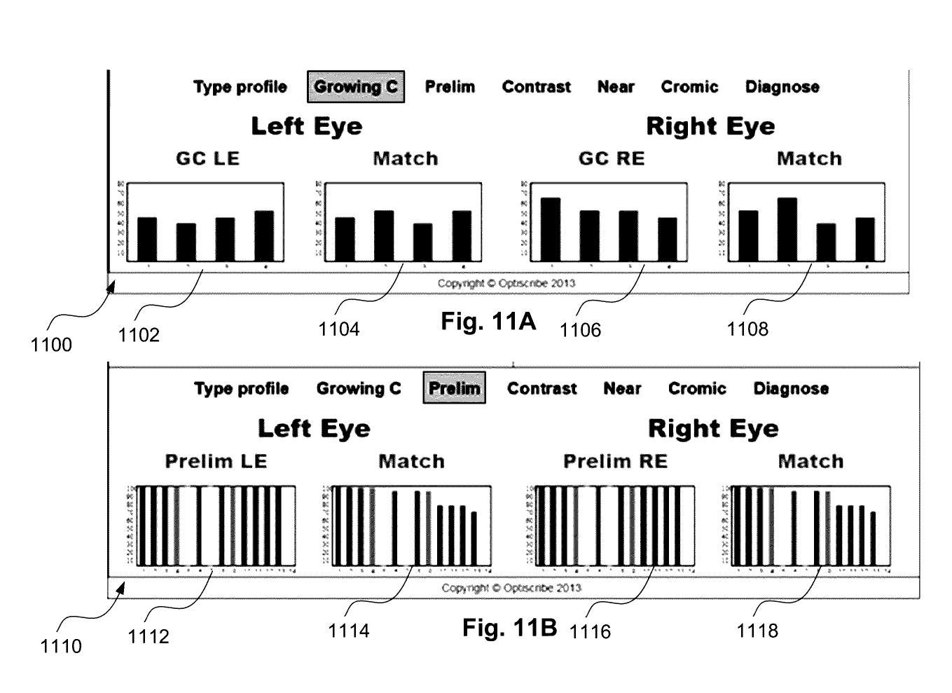

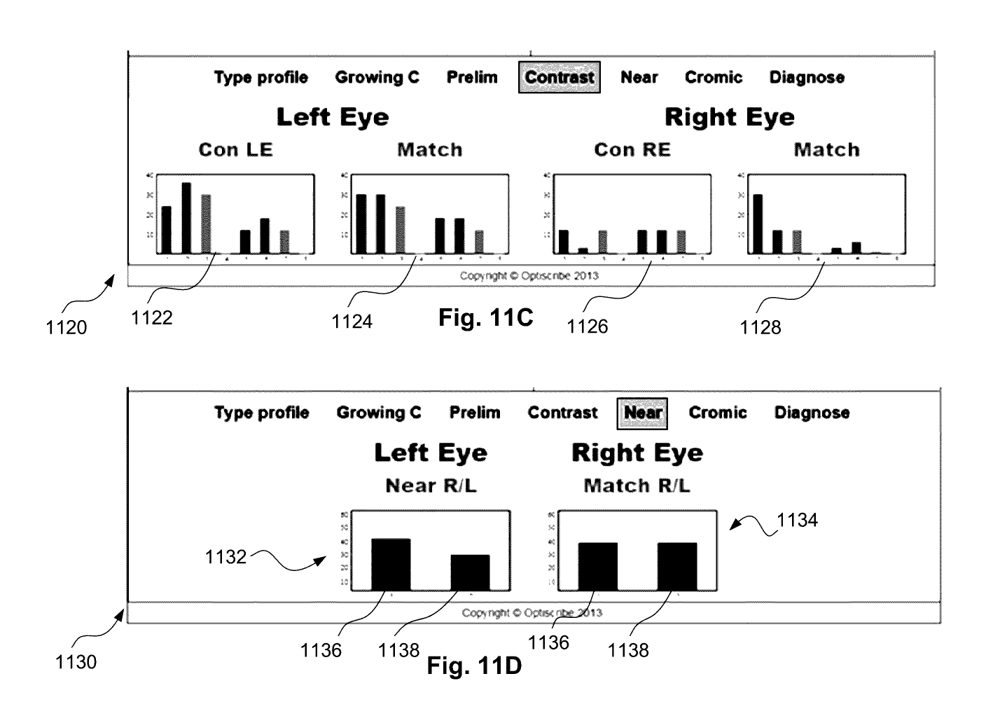

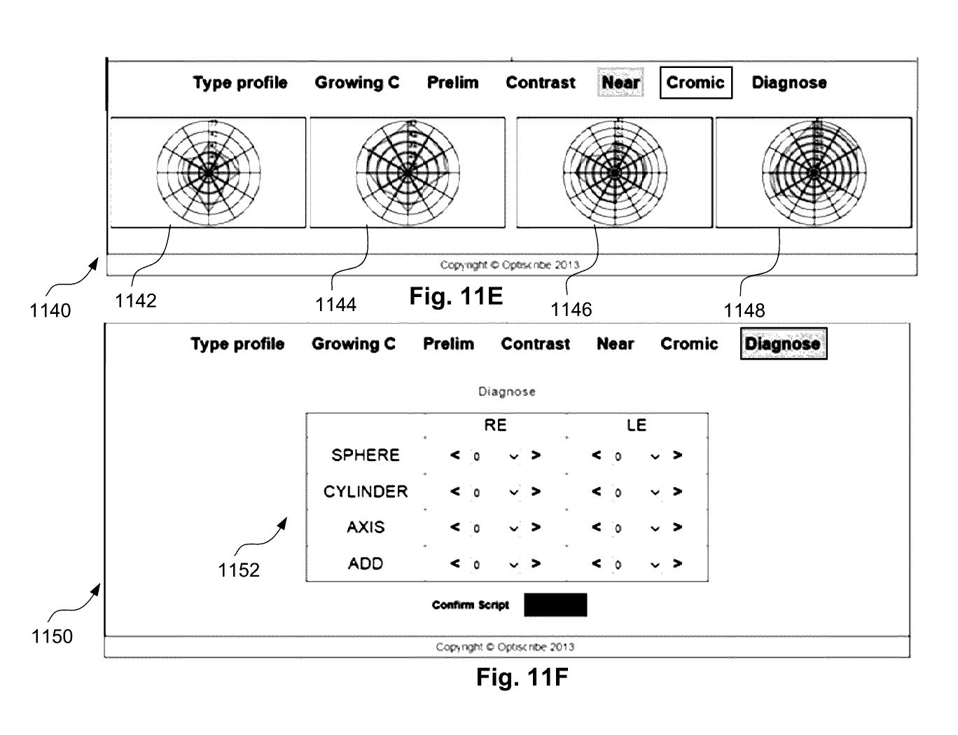

(i) Big C test data 204, obtained as described with reference to FIGS. 6 and 11 and the test data 517 of the aforementioned Publication WO 02/00105;

(ii) Chromo test data 212, obtained as chromatic Astigmatism test, as described in FIG. 21 of the aforementioned Australian Patent Application No. 2014904932;

(iii) Contrast Pattern test data 208, obtained as described from the Contrast Pattern test 525 of the aforementioned Publication WO 02/00105;

(iv) White Visual Acuity Test data 206, obtained as described from the Growing C Test 512 of the aforementioned Publication WO 02/00105;

(v) Blue Colour Contrast Pattern Test data 209, obtained as described from the Contrast Pattern Test 525, performed using blue coloured patterns, of the aforementioned Publication WO 0200105; and

(vi) Near test data 210, obtained as described in the aforementioned Publication WO 02/00105 from a test for near visual acuity test data 529.

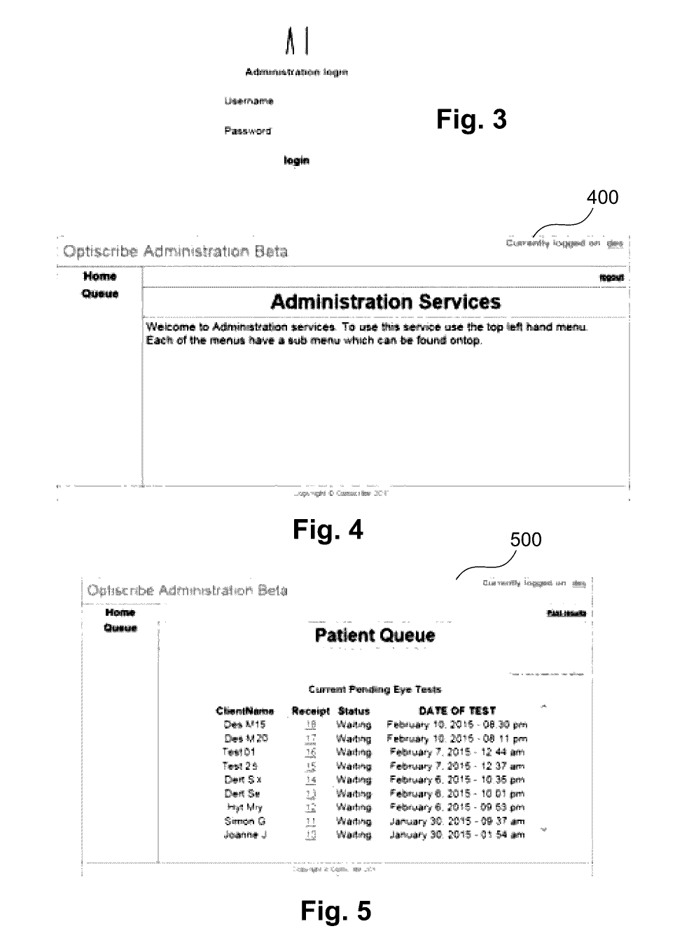

The program 200 operates through a series of GUI screens which interface to the analytical program 133. A first of those screens is encountered at a login step 216 as seen in FIG. 3 which affords a user access to the analytical program with password entry. Upon access, the user is presented with a directory 400 at step 218 as seen in FIG. 4. The directory affords the user access to parts of the program 200.

At step 220, the user can access a patient queuing page 500, as seen in FIG. 5. The queue list 500 shows the name and other details of patients, whether a diagnosis had been completed, test date, and the name of the analyst (i.e. the “user” as described herein).

FIG. 6 shows a screen shot of analysis interface GUI page 600 that is used in a number of the steps of the method 200.

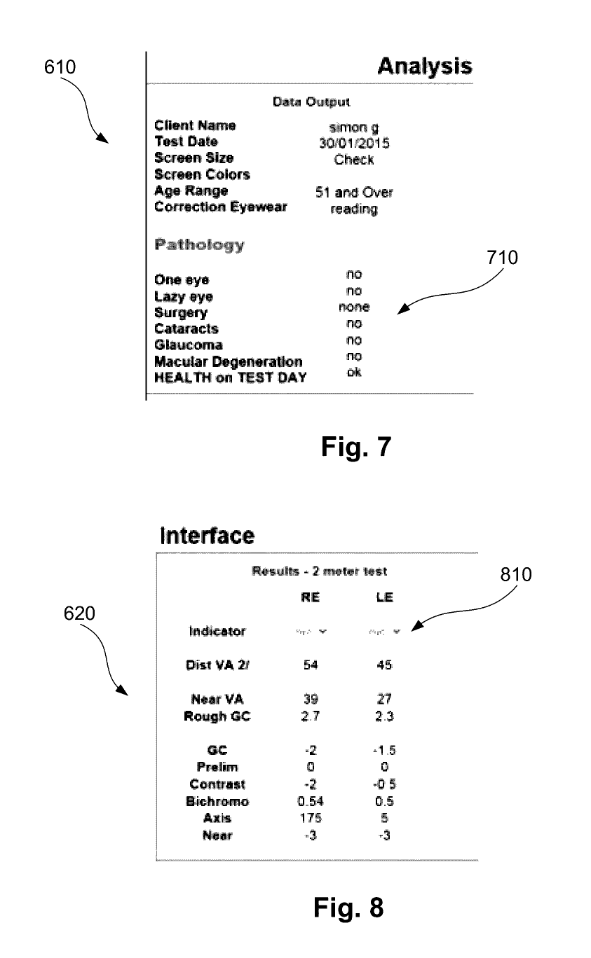

The analysis interface page 600 has a top-left Data Output window 610, also seen in FIG. 7, showing:

(i) client details and date confirms the patient being tested:

(ii) Screen Check validates that the screen monitor image used in the vision test was acceptable;

(iii) Screen Colours validates that the screen image colours were acceptable;

(iv) Age range indicates the age range of the patient; and

(v) Correction Eyewear indicates what sort of eyewear patient is currently using.

The Data Output window 610 also has a Pathology Section indicates whether the patient has stated that he/she has a pathological condition that might invalidate or affect the test results. Such conditions might be single eye, lazy eye, eye surgery, cataracts, glaucoma, macular degeneration, and whether the patient's eyes feel well enough to have the test.

The analysis interface page 600 of FIG. 6 also has a top-right window 620, seen in more detail in FIG. 8, which displays the Results of the 2 metre Test for right eye (RE) and left eye (LE). These test results include:

Indicator—this is an automatically calculated assessment of the patient's vision refractive type and severity based upon an analysis of the patient's question results data 214 and is used to categorise whether the patient is a large myopic, myopic, neutral, hyperopic, large hyperopic, hyperopic astigmatism, myopic astigmatism, hyperopic pathology, myopic pathology, mid pathology and other possible pathology combinations.

Dist VA 2/—this gives a computed 6 m distance visual acuity value for the right and left eyes.

Near VA—this gives a computed 40 cm near visual acuity value for the right and left eyes.

Rough GC—this gives a reasonably accurate total power value in Dioptres for right and left eyes, determined from a rough growing C test.

GC—this gives a calculated power value in dioptres corrected for vision type (e.g. myopia) for right and left eyes, determined from a detailed growing C test.

Prelim—this gives an estimated power value in dioptres based upon the prelim test for right and left eyes.

Contrast—this gives a calculated power value in Dioptres corrected for vision type for right and left eyes.

Bichromo (Cromic) Test—this gives a calculated power value in dioptres for the astigmatism of the right and left eyes.

Axis—This gives calculated astigmatic angle value in degrees for the right and left eyes.

Near—This gives a calculated near power value in dioptres corrected for vision type for right and left eyes.

The analysis interface page 600 of FIG. 6 also has a Bottom Window Buttons 630 which are selectable via the GUI using the mouse 103 for example to trigger various graphic displays, in a Bottom Window 640 of the interface 600, of the data shown Top Right window 620 compared with data from a reference data base.

FIG. 9 shows detail of the bottom window 640 for which, in this example, the user has selected display of a Type Profile from the buttons 630. The type profile formed of two graphs in which a left graph 910 shows graph of patient data for both eyes combined, and an adjacent right graph 920 shows an automated match of an indicator pattern to the left graph 910 according to one of a number of type profiles. An option of the user selecting a manual match can be used to verify the best fit if required. Exemplary indicator pattern type profiles that are used for matching are shown in plots or graphs of FIGS. 10A-10I and include big hyperope (FIG. 10A), hyperope (FIG. 10B), mid (FIG. 10C), myope (FIG. 10D), big myope (FIG. 10E), astigmatic myope (FIG. 10F), astigmatic hyperope (FIG. 10G), pathological hyperope (FIG. 10H), and pathological myope (FIG. 10I).

The horizontal units of FIGS. 10A to 10I are four indices, one each for hyperopia, normality, myopia and severity, shown graphed from left to right in the graphical representation.

The algorithm used to calculate the indices is based on a statistical study of the diagnostic importance of the patient's answers 214 to a graded set of visual history questions which are presented to the patients in common language readily understood by the patient that takes into account their ethnicity and likely level of knowledge. The questions were rated as to whether the answers would normally indicate hyperopia, normality or myopia. The responses were summed to give an indication of severity.

Each of the indices where calculated as sums of the positive answers to question known to be related to those refractive states.

The vertical axes in FIGS. 10A to 10I are scaled to a maximum of 10 for each of the indices based upon the maximum number of positive responses possible in the client history questionnaire study.

FIG. 11A shows an example where the user has selected “Growing C” from the buttons 630. This selection results in the display 1100 of four graphs, two each for the left and right eyes, as shown. Graphs 1102 and 1106 on the left show the values of the patient data from the Growing C test for the left and right eyes. Graphs 1104 and 1108 show matching data from the reference indicator pattern database 190 of Growing C patient data. The vertical axis of FIG. 11A is scaled in screen pixels and the horizontal axis refers to the four orientations of the growing C image, which are left, up, right, down.

FIG. 11B shows an example where the user has selected “Prelim” from the buttons 630. This selection results in the processor 105 displaying on the display 114 four graphs 1110, two each for the left and right eyes. Graphs 1112 and 1116 on the left show the values of the patient data from the Prelim test for the left and right eyes. Graphs 1114 and 1118 show matching data from the reference indicator pattern database 190 of Prelim patient data. The vertical axis of FIG. 11B is scaled in contrast from 0 to 100 and the horizontal axis refers to the frequencies of the test patterns ranging from the left being 1 cycle to the right equal to 30 cycles. In some cases the frequencies where tested in up to four orientation vertical, horizontal, diagonal right and diagonal left.

FIG. 11C shows an example where the user has selected “Contrast” from the buttons 630. This selection results in the processor 105 displaying on the display 114 four graphs 1120, two each for the left and right eyes. Graphs 1122 and 1126 on the left show histogram values of the patient data from the Contrast test for the left and right eyes. Graphs 1124 and 1128 show matching data from the reference indicator pattern database 190 of Contrast patient data. The vertical axis of FIG. 11C is scaled in highest frequency seen from 1 to 36 cycles and the horizontal axis refers from left to right four orientations of grey patterns followed by four orientations of blue patterns. The four orientations were varied from a set of vertical horizontal and diagonals right and left.

FIG. 11D shows an example where the user has selected “Near” from the buttons 630. This selection results in the processor 105 displaying on the display 114 a representation 1130 including two graphs, one for each of the left and right eyes, 1132 and 1134 respectively. The left side data 1136 of the graphs show histogram values of the patient data from the Near test, while the adjacent right sides 1138 show matching data from the reference indicator pattern database of Near patient data. The vertical axis of FIG. 11D is scaled in screen pixels and the horizontal axis refers to the near calculated value compared its match on the right taken from the clinical data base for each eye.

FIG. 11E shows an example where the user has selected “Chromic” from the buttons 630. This selection results in the processor 105 displaying on the display 114 four radial graphs 1140, two each for the left and right eyes. Graphs 1142 and 1146 from the left side show the values of the patient data from the Chromo test for the left and right eyes. Graphs 1144 and 1148 show matching data from the reference indicator pattern database of Chromo patient data.

The radial axis of FIG. 11E is scaled in degrees while the radius from the centre is scaled in screen pixels of the average separation distance between the red and blue bars for angular orientation.

FIG. 11F shows an example where the user has selected “Diagnose” from the buttons 630. This selection results in the processor 105 causing the display 114 to display a representation 1150 of the final diagnosis entry as determined by the processor 105 analysing the matching of data represented by the foregoing displays to the user. As seen the prescription is presented in the form of a table 1152 with columns for each of the right eye (RE) and left eye (LE) and rows for each of Sphere (spherical power component), Cylinder (cylindrical power component), Axis (axial component), and Add (additional optical power for near reading component). Thus the user/diagnostician can then review this prescription based upon the presented information, manually modify it if necessary and then select one of two buttons, “Confirm Script” in which the prescription is confirmed and assigned to the patient record, or “Fail” where no script is issued or recorded against the patient. This can suggest erroneous data.

Returning to FIG. 2, a step 222 is then carried out using the data displayed in the Top left window 610 of FIG. 6. This data is replicated in FIG. 7. With this information, the user of the analysis program 133 is able to:

(i) verify patient details.

(ii) assess whether calibration for vision testing, as described in Publication No. WO 02/00105, has been checked. If correct the calibration is correct, the user can proceed with the analysis. If the calibration is not correct, then the user can then suspect accuracy of test data for the analysis:

(iii) assess the age range of the subject to see if there exists a pathology risk or if age outside test specified range (either too young or too old—age is known to influence accommodation problems):

(iv) ascertain whether the patient is already wearing corrective eyewear, as this information will give a clue to what type of refraction problem.

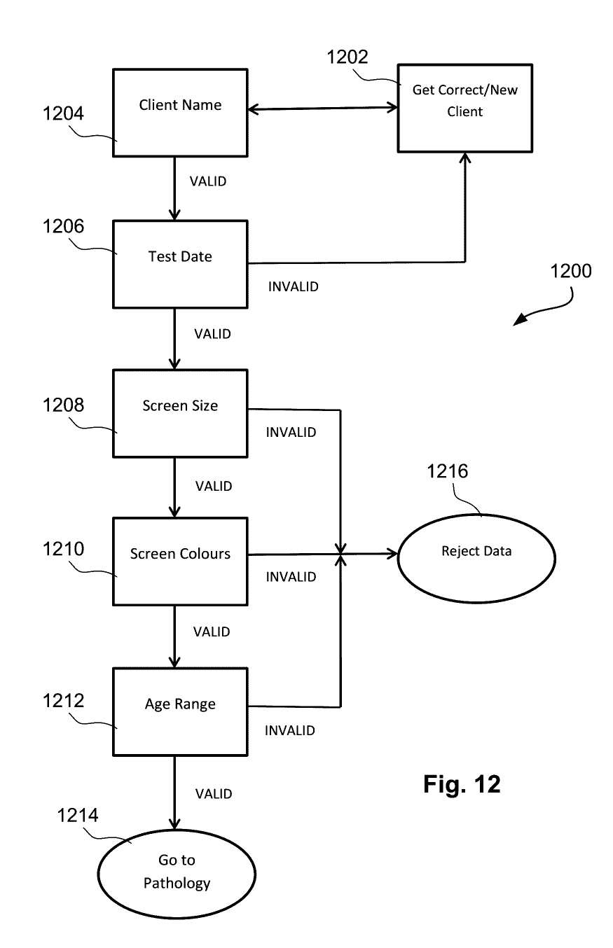

The processing of step 222 is shown in detail in a preferred sub-process 1200 of FIG. 12. At step 1202, the processor 105 gets new client data and at step 1204 confirms the client's name. Step 1206 checks the test date. Where the test date is older than a predetermined threshold (e.g. 4 weeks) the data is considered invalid, and the process returns to step 1202. Where the date is valid, the process 1200 proceeds to check each of screen size 1208, screen colours 1210 and age range 1212. Where test data checked in any of these tests fails, the entire data set for the patient is rejected at step 1216. Where all the data checks correctly, step 1214 proceeds to the pathology checks of step 224.

Next, the method 200 proceeds to step 224 where a pathology check is carried out using the data 710 displayed in bottom of Top left window 610. In step 224, a check is performed by the diagnostician to determine if any pathology that may affect clarity of eyes or reliability of results.

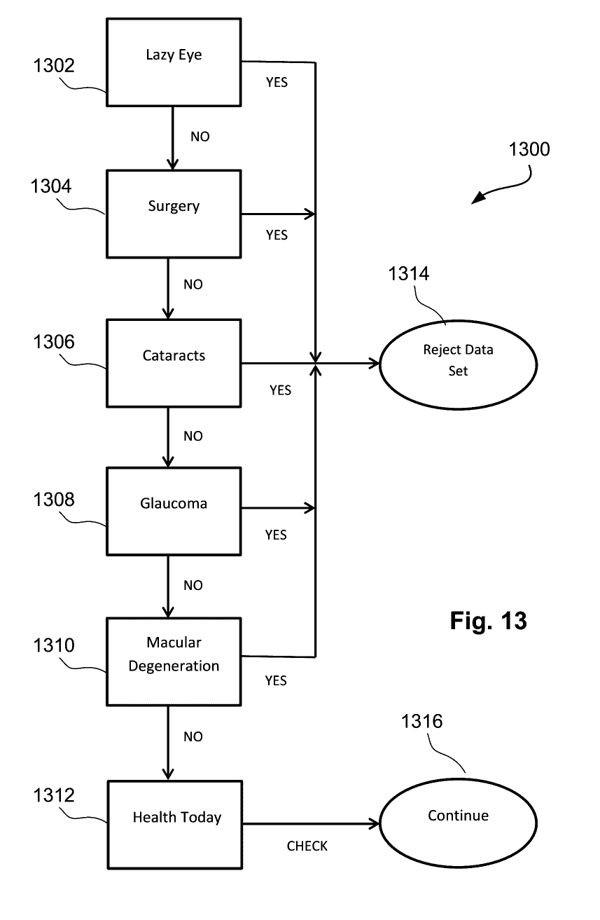

Detail of step 224 is shown in FIG. 13 for a process 1300 where the patient answers 214 are checked by the processor 105. In a preferred implementation, as illustrated, positive answers for each of Lazy eye 1302, Surgery 1304, Cataracts 1306, Glaucoma 1308, and Macular Degeneration 1310 will cause the process 1300 to reject the data set at step 1314. Those pre-existing conditions necessitate direct and specialized evaluation of the patient by an optometrist and automated testing is not suitable for the patient. A final check 1312 assesses the patient's health on the day of actual testing, which does not obviate further automated analysis and diagnosis, but where such is sub-optimal, can flag the diagnosis for additional review by the diagnostician user.

For example, with Lazy eye, sight will be reduced not because of the optical power of eye. Also, cataracts operate to reduce the amount of light entering the eye and so will reduce the clarity of the image resulting in reduced contrast.

Health on test day—if any fatigue conjunctivitis, dry eye, that may create or film over the eyes or too tired the give reliable results. The patient may be asked to redo test or second time to cross check or when feeling better. The health question is conveniently located in the GUI display 630. Since such is not strictly part of the pathology tests, such may be displayed elsewhere.

Where the pathology of the patient is unacceptable or unreliable, the pathology check 224 will fail and the method 200 ends, thereby preventing completion of the test and the prescription of potentially unreliable spectacles.

Where the pathology test 224 is passed, the method 200 proceeds to step 226 to determine and display the refractive type for the patient. This step is carried out using the data displayed in FIG. 4 Top Right window 620, also seen in FIG. 8. When the input data set is found to be internally consistent and validity checks found to be acceptable then it is possible for the processor 105 to ascertain and display the refractive type.

As seen in the window 620, shown in detail in FIG. 8, the Indicator stages the analysis into two main streams. The streams and components thereof are selectable by way of drop-down menus 810, selectable by the user to confirm that the indicator pattern was the best match considering the refractive type. The Indicator stage of the menus 810 are each automatically selected by the program 133 but may be subsequently manually selected by the user for manual checking where desired or necessary. A high reliability stream labelled hyperopic, mid, and myopic, and a lower reliability stream with several designations such as mid and high myopia, mid and high hyperopia, Astigmatism, myopic astigmatism, hyperopic astigmatism.

Normally the high reliability stream or selection causes the processor 105 via the program 133 to automatically analyse the appropriate data parameters from the data stream.

As discussed above, this is performed by the processor 105 comparing the analysed data of refractive type for the current patient against a database of indicator patterns for refractive type of previously optometrically assessed patients and their vision data covering a wide range of optical conditions. Where a match or substantial match is found, the matching optometrically assessed refractive type can be assigned to the current patient.

After determination of the refractive type in step 226, the method 200 proceeds to step 228 which implements a consistency analysis carried out using the data displayed in Top Right window 620 (FIG. 8).

In step 228 the user (analyst) utilises the data which is displayed in conventional visual acuity terminology to allow the analysis to relate the visual acuity to a conventional diagnosis using a standard image size acuity calculation of size relative to distance. The user can then determine whether the machine analysis is consistent with the expected acuity and the displayed refractive value.

A 6/6 visual acuity (VA) reading is considered normal vision, a 6/12 VA would normally relate to an optical power of −0.5 dioptre to +1.5 dioptre of cylindrical power, while a VA of 6/36 would relate to an optical power of −2 dioptres or a −4 dioptres of cylindrical power.

The method 200 then proceeds to step 230, where an examination of the major refractive power indicator is performed. This relates to a rough assessment of the Growing C test (Rough GC). This is a display, as seen in FIG. 11A, of the total spherical optical power for each eye uncorrected for refractive type. As seen in FIG. 11A, the graphical representation of the patient's data (GC LE, GC RE) against corresponding Match data allows the user analyst to get a quantitative view of the patient's maximum likely refractive status. The Growing C test is the major spherical optical power indicator used within the analysis. It allows the diagnostician to get a qualitative view of the patient's refractive status.

The program 133 again uses corresponding indicator patterns to channel the analysis into two main streams. The present inventors have found that where a client is in Group A indicates that is a high reliability group which have been found by experiment to be almost always correct. In the case of the highly reliable stream of Group A it verifies that the results are within the highly reliable range of +/−1.5 Dioptres.

If the program 133 indicates that the client's data is in the less reliable Group B then this indicates that the diagnostician will need to review more carefully any diagnosis made by the program 133, examining the match between data sets and determine whether a manual correction is necessary. In the case of the less reliable stream of Group B, which will normally fall within the ranges −4 to −1.5 and +1 to +4 dioptres, the Rough GC gives the initial indication of the refractive power and allows the diagnostician to more closely supervise the analytical results by reviewing the full data output presented to the diagnostician as graphic displays matched to the best match with similar patient data sets drawn from a data base of results where the diagnosis has been confirmed in previous clinic studies by classical methods.

If the test was carried out in the presence of pathology, the GC will give an estimate of the patient's optical power however, further diagnosis is normally blocked and no further diagnosis carried out.

Failure on the Big C test, which is the first of the set of vision tests carried out by the system of the aforementioned International Patent Publication No. WO 02/00105, will normally indicate whether the patient has a refractive status outside the +/−4 dioptre vision test accuracy range. If allowed the test may have been continued at a working distance of 1 metre instead of 2 m. In this case the test accuracy can be extended to +/−6 dioptres and the 1 metre set of algorithms used instead of the 2 metre set. In this case the rough GC will still give an accurate optical power.

In some implementations, it may be necessary to verify the power value for each eye. Here, the user/analyst may examine the individual data outputs with best matched data from a clinical studies data base. This is depicted in FIG. 2 as step 232.

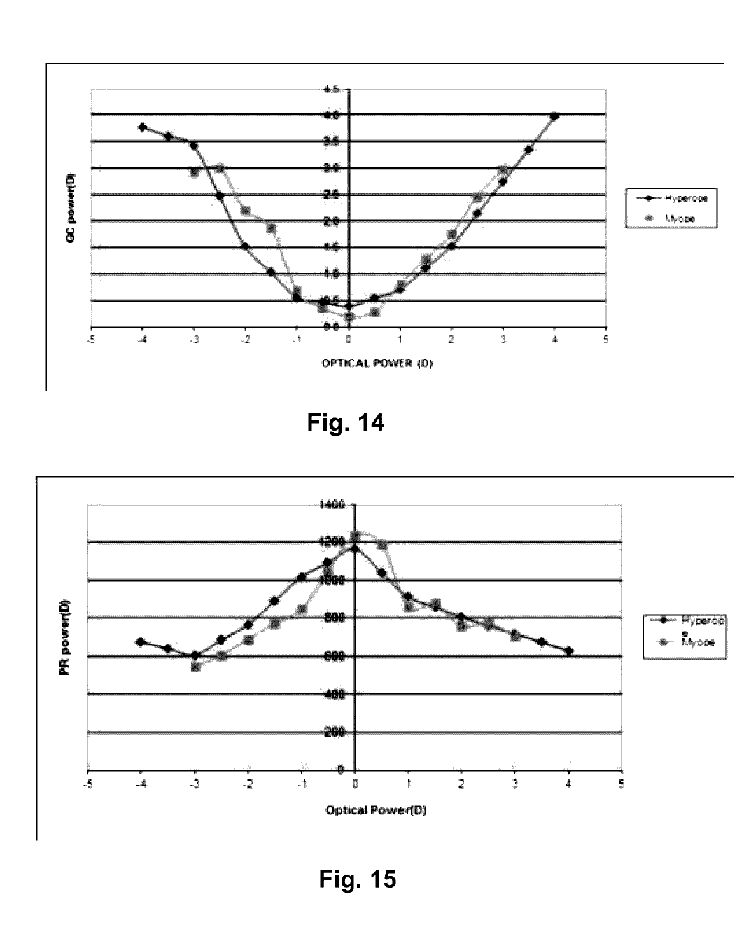

The Growing C, the Prelim acuity test and the Blue Contrast test all can be used to give estimates of the visual power in dioptres, but each are affected by different sub-clinical affects. The Growing C gives a good estimate of the total optical power which is the combined power of the spherical and cylindrical components largely unaffected by the angle of astigmatic axis. A typical Growing C plot is shown in FIG. 14 for Growing C power of two patients exhibiting hyperope and a myope.

The Prelim and Blue contrast tests give an accurate estimation of the total power at low cylindrical powers but are affected by the angle of astigmatic axis to an increasing degree as the cylindrical power increases. Thus a power derived from a Growing C estimation can act as the maximum possible power and, by subtraction, the powers from the Prelim contrast and Blue contrast tests indicate the relative power of the cylindrical component.

A typical Prelim Power curve is shown in FIG. 15 for Prelim Contrast for 2 patients exhibiting hyperope and a myope.

The Chromo test gives both an output of spherical power, cylindrical power and astigmatic angle.

The spherical power estimated by the Chromo test should be similar to the estimations of the powers based on the previous tests and lower as the cylindrical power increases. The cylindrical power can be cross checked for magnitude using the difference on power between the Growing C and Prelim contrast tests.

It can be seen from FIG. 15 that there is some small variability between the hyperope and myope plots which is normal as each individual's visual system is unique to them and slightly different from others. Consequently it is difficult to get accurate powers based on single numeric indexes taken from the vision test outputs. The solution found by the present inventors is to match data sets of Growing C, Prelim contrast and Blue Contrast and Chromo tests with similar sets derived from clinical studies of known patients stored in a database kept for the purpose.

For the purposes of matching a least squared algorithm was used to determine the best match.

Hence in summary the diagnostician may examine the individual data output sets with best matched data from a clinical studies data base. In this case:

(a) GC, Prelim, Contrast give a redundant display of the spherical power value for each eye as determined by the appropriate algorithms. This improves the accuracy and confidence in the diagnosis as similar results are obtained from each different test.

(b) Prelim—is a contrast test that not only gives an estimation of optical power for each eye but also allows the Astigmatic angular component of the astigmatic power to be estimated and verified;

(c) Bichromo gives both a spherical and astigmatic power which is subtractive allowing estimation of the astigmatic power independent of the spherical power; and

(d) Near gives an optical power for Near sight test which is used to prescribe the reading add power.

When step 230 has been performed, and step 232 where applicable, the method proceeds to step 234 to prescribe the lens power, thereby forming the optical prescription for the patient. Selection of the Diagnose button 620 (FIG. 11F) by the user causes display of the Lens prescription format. In the highly reliable cases, generally considered to be +/−1.5 dioptres optical power, the table 1152 is expected to be filled in automatically by the processor 105 from the automated analysis. In all cases outside +/−1.5 dioptre range, the vision professional (user) reviewing the case is required to fill in the details manually to ensure that the analysis is as accurate as possible.

In each of these instances, a well-trained user diagnostician can perform a rapid visual comparison between the graphically represented test data of the current patient against corresponding graphically displayed matching data obtained from the database 190 of previously optometrically assessed patient vision data covering a wide range of optical conditions. This visual comparison is used to thereby confirm matching optical parameters and thus confirm an optical prescription for the current patient. Where the user diagnostician is uncertain of the accuracy of a match, for example because of apparently conflicting test data, the diagnostician can reject the diagnosis and order further testing.

### INDUSTRIAL APPLICABILITY

The arrangements described are applicable to the computer and data processing industries and particularly for the substantially automated analysis and determination of optical lens prescription for patients having undergone automated vision testing.

The foregoing describes only some embodiments of the present invention, and modifications and/or changes can be made thereto without departing from the scope and spirit of the invention, the embodiments being illustrative and not restrictive.

The invention is claimed as follows:

1. A computer-implemented method for vision diagnosis for lens prescription comprising:
retrieving vision test data recorded from a computer assisted vision test of a patient;
assessing the vision test data of the patient for suitability for vision diagnosis;
analyzing the vision test data of the patient to display a representation of at least a part of the patient data;
matching the analyzed part of the patient data with corresponding data from previously optometrically assessed and diagnosed vision data covering a wide range of optical conditions; and
establishing a diagnosis of the vision of the patient from the matching.

2. A method according to claim 1, wherein the assessing comprises validating patient data and validating patient pathology such that where either such validation fails, the method terminates.

3. A method according to claim 2, wherein validating the patient data comprises validating data associated with the capture of the vision test data of the patient, said capture data being at least one of test date, screen size, screen colours, and age range.

4. A method according to claim 2, wherein validating patient pathology comprises assessing patient answers to qualitative questions regarding at least one of lazy eye, surgery, cataracts, glaucoma and macular degeneration.

5. A method according to claim 1, wherein the matching comprises displaying a representation of a corresponding part of the previously optometrically assessed and diagnosed patient data with the displayed representation of the analyzed part of the patient data.

6. A method according to claim 5, wherein the analyzing and matching comprises displaying, in a graphical user interface, adjacent representations of analyzed current patient data and previously optometrically assessed and diagnosed patient data for at least one indicator pattern associated with the vision test data.

7. A method according to claim 6, wherein the indicator pattern is a type profile having matching types selected from the group consisting of big hyperope, hyperope, mid, myope, big myope, astigmatic myope, astigmatic hyperope, pathological hyperope, and pathological myope.

8. A method according to claim 6, wherein the indicator pattern is associated with a Growing C test.

9. A method according to claim 6, wherein the indicator pattern is associated with a Prelim test.

10. A method according to claim 6, wherein the indicator pattern is associated with a Contrast test.

11. A method according to claim 6, wherein the indicator pattern is associated with a Near test.

12. A method according to claim 6, wherein the indicator pattern is associated with a chromic test.

13. A method according to claim 6, wherein the establishing of the diagnosis comprises displaying in the graphical user interface values for at least each of spherical power, cylindrical power, and astigmatic angle, for each patient eye.

14. A method according to claim 13 further comprising displaying in the graphical user interface a value for additional optical power for near reading component for each patient eye.

15. A method according to claim 1, wherein the vision test data comprises computer assisted vision test data of a plurality of patients, said method further comprising dividing the test data associated with the patients into a plurality of groups based upon measured values of visual acuity, and processing patient data associated with each group as a stream.

16. A method according to claim 15, wherein:
a first group comprises patients with visual acuity in the range −1.5 to +1.5 dioptres, and who have only minor astigmatism, generally less than 0.5 dioptres;
a second group comprises patients with visual acuity in the range −1.5 to −4 and +1.5 to 4 dioptres; and
a third group comprises patients with visual acuity problems outside the range −1.5 to −4 and +1.5 to 4 dioptres.

17. A system for assisted vision diagnosis for lens prescription, comprising:
a database of previously optometrically assessed and diagnosed vision data covering a wide range of optical conditions establishing a plurality of visual indicator patterns;
a vision test data set associated with a patient recorded from a computer assisted vision test of the patient;
a processor associated with a program, the program being executable by the processor to:
retrieve the vision test data of the patient;
assess the vision test data set of the patient for suitability for vision diagnosis;
analyze the vision test data set of the patient to display a representation of at least a part of the patient test data;
match the analyzed part of the patient data with corresponding data from the database of previously optometrically assessed and diagnosed vision data; and
establish a diagnosis of the vision of the patient from the matching.
